(19) United States
(12) Patent Application Publication
(10) Pub. No.: US 2016/0123941 A1
(43) Pub. Date: May 5, 2016

(54) REMOTE CHEMICAL DETECTION AND IDENTIFICATION SYSTEMS AND METHODS

(71) Applicant: Elwha LLC, Bellevue, WA (US)

(72) Inventors: Jeffrey A. Bowers, Bellevue, WA (US); Alistair K. Chan, Bainbridge Island, WA (US); Roderick A. Hyde, Redmond, WA (US); Muriel Y. Ishikawa, Livermore, CA (US); Jordin T. Kare, Seattle, WA (US); Charles Whitmer, North Bend, WA (US); Lowell L. Wood, JR., Bellevue, WA (US)

(73) Assignee: ELWHA LLC, Bellevue, WA (US)

(21) Appl. No.: 14/530,165

(22) Filed: Oct. 31, 2014

(51) Int. Cl. G01N 33/00

(52) U.S. Cl. CPC G01N 33/0009

(57) ABSTRACT

A system for detecting the release of a chemical substance includes a release device configured to release a chemical substance into a surrounding environment based on receipt of a control signal; a signaling device configured to transmit the control signal to the release device; and a sensor device configured to detect the chemical substance in the surrounding environment based on an analysis of the surrounding environment.

### BACKGROUND

Various systems exist for remotely detecting and identifying items, persons, and the like. For example, radio frequency identification (RFID) tags may be used as tracking tags and attached to objects. A transceiver sends a radio frequency signal to the RFID tag, and receives a corresponding response. Based on the response, the object can be identified.

### SUMMARY

One embodiment relates to a system for detecting the release of a chemical substance. The system includes a release device configured to release a chemical substance into a surrounding environment based on receipt of a control signal; a trigger device configured to transmit the control signal to the release device; and a sensor device configured to detect the chemical substance in the surrounding environment.

Another embodiment relates to a system for detecting the release of a chemical substance. The system includes a trigger device configured to transmit a control signal to a release device; and a sensor device configured to sense the presence of a chemical substance released from the release device in the surrounding environment.

Another embodiment relates to a system for identifying items. The system includes a release device configured for attachment to an item and to release a chemical substance into a surrounding environment based on receipt of a control signal; a trigger device configured to transmit the control signal to the release device; a sensor device configured to acquire substance data regarding the presence of the chemical substance in the surrounding environment; and a processing circuit configured to identify the item based on the substance data.

Another embodiment relates to a method of detecting the presence of a chemical substance. The method includes wirelessly transmitting a control signal to a release device; releasing a chemical substance from the release device into a surrounding environment based on the control signal; and detecting the presence of the chemical substance in the surrounding environment.

Another embodiment relates to a method of detecting the presence of a chemical substance. The method includes transmitting a control signal to a release device using a trigger device; and sensing the presence of a chemical substance released from the release device in the surrounding environment.

Another embodiment relates to a method of identifying items. The method includes coupling a release device to an item, the release device configured to release a chemical substance into a surrounding environment based on receipt of a control signal; transmitting the control signal to the release device using a trigger device; acquiring substance data regarding the presence of the chemical substance in the surrounding environment using a sensor device; and identifying the item based on the substance data.

Another embodiment relates to a release device for releasing a chemical substance as a signal, including a substrate; a chamber configured to store a plurality of chemical substances; and a control system configured to selectively control release of each of the plurality of chemical substances into a surrounding environment based on receipt of an input.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic representation of a system for triggering and detecting the release of chemical substances according to one embodiment.

FIG. 2 is a schematic representation of the system of FIG. 1 shown in greater detail according to one embodiment.

FIG. 3 is a top view of a release device according to one embodiment.

FIG. 4 is a cross-section view of a portion of a release device according to one embodiment.

FIG. 5 is a cross-section view of a portion of a release device according to another embodiment.

FIG. 6 is a cross-section view of a portion of a release device according to another embodiment.

FIG. 7 is a schematic representation of a sensor device according to one embodiment.

FIG. 8 is a schematic representation of a sensor device according to another embodiment.

FIGS. 9A-9B are illustrations of various types of data stored for use in connection with the system of FIG. 1 according to alternative embodiments.

FIG. 10 is a block diagram illustrating a method for detecting the release of a chemical substance according to one embodiment.

FIG. 11 is a schematic illustration of various sensing devices usable to detect the presence of chemical substances released by a release device according to one embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Referring to the figures generally, various embodiments disclosed herein relate to systems and methods for releasing and detecting various chemical substances. For example, in some embodiments, a trigger device (e.g., a wireless transceiver, a signaling device, etc.) transmits a control signal such as a radio frequency (RF) signal. A release device, such as a tag or similar device having a storage chamber or reservoir of one or more substances, is attached to an object, person, etc., and receives the RF signal. In response to the control signal, the release device releases one or more chemical substances (e.g., fluid substances such as gases) to a surrounding environment (e.g., the surrounding air, a surrounding liquid, etc.). A sensor device can then analyze a fluid sample of the surrounding environment and determine the presence or absence of one or more possible released substances. As such, by sampling the surrounding environment, the sensor device can detect the presence of persons or objects with release devices that release substances in response to the control signal. Additionally, the sensor device may also detect a state or condition determined by the release device, spatial or temporal information regarding timing of the release, distance and direction to a potential point of release, a degree of enclosure of a release device (e.g., whether the release device is exposed, fully or partially enclosed (e.g., covered, wrapped, etc.), and the like).

It should be noted that as used herein, to receive a signal at a release or similar device in some embodiments means that the release device is within range of the signal such that the signal triggers an appropriate response (e.g., a release of a chemical signal, etc.). To receive the signal may, but does not necessarily, mean to receive data provided by the signal. For example, receipt of a control signal may include stimulation of a magnetostrictive resonator or similar device at the release device by way of passage of the signal past the release device.

The features discussed herein may be implemented in the context of tracking items where more conventional radio frequency identification (RFID) technologies may be insufficient or inappropriate to provide reliable tracking. For example, in areas where RF signal transmissions from an RFID tag may be unreliable (e.g., jammed, intentionally or unintentionally screened, etc.), a substance release device may provide a more reliable method of tracking In some embodiments, a release device can include both radio frequency signaling/emissions and chemical release features integrated into a single device.

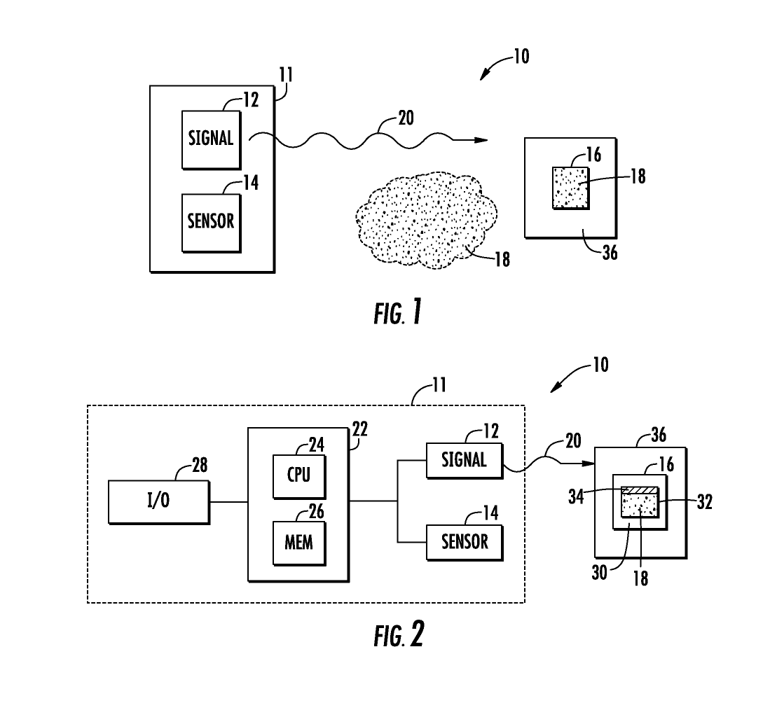

Referring now to FIG. 1, system 10 for releasing and detecting one or more substances (e.g., chemical substances) is shown according to one embodiment. System 10 includes detection system 11 and release device 16. Detection system 11 includes trigger device 12 and sensor device 14. Release device 16 is configured to contain substance 18 (e.g., one or more chemical substances) and be attached or coupled to attachment surface 36 of an item, person, etc. In operation, trigger device 12 emits control signal 20. When release device 16 is close enough to trigger device 12 to receive control signal 20, release device 16 releases substance 18 into the surrounding environment. Sensor device 14 analyzes fluid samples (e.g., gas or liquid) from the surrounding environment, such that sensor device 14 can detect whether substance 18 is released from release device 16 and identify the presence of one or more chemical substances. The identification of the presence of one or more chemical substances provides a chemical signature that may in turn be associated with various items, persons, etc.

Referring further to FIG. 1, system 10 can take a variety of configurations. For example, various components shown in FIG. 1, such as trigger device 12 and sensor device 14, can be integrated into a single device. Alternatively, various components can be provided as separate components and/or located remotely relative to each other. Furthermore, system 10 can find application in a wide variety of environments, including public facilities, security areas, and the like. Further yet, the surrounding environment can be a gaseous environment, a liquid environment, a low pressure or vacuum environment, or a combination thereof. The chemical substances released by the release device are generally volatile in gaseous/vacuum environments and/or soluble in liquid environments.

Referring now to FIG. 2, system 10 is shown in greater detail according to one embodiment. Detection system 11, in addition to trigger device 12 and sensor device 14, includes processing circuit 22 and input/output device 28. In one embodiment, processing circuit 22 is configured to control the operation of trigger device 12 and sensor device 14 and to provide outputs to and receive inputs from device 28.

As shown in FIG. 2, processing circuit 22 includes processor 24 and memory 26. Processor 24 may be implemented as a general-purpose processor, an application specific integrated circuit (ASIC), one or more field programmable gate arrays (FPGAs), a digital-signal-processor (DSP), a group of processing components, or other suitable electronic processing components. Memory 26 is one or more devices (e.g., RAM, ROM, Flash Memory, hard disk storage, etc.) for storing data and/or computer code for facilitating the various processes described herein. Memory 26 may be or include non-transient volatile memory or non-volatile memory. Memory 26 may include database components, object code components, script components, or any other type of information structure for supporting the various activities and information structures described herein. Memory 26 may be communicably connected to processor 24 and provide computer code or instructions to processor 24 for executing the processes described herein.

Processing circuit 22 controls operation of trigger device 12 and sensor device 14. For example, processing circuit 22 may control the timing (e.g., time and duration) of the delivery of control signal 20. In one embodiment, processing circuit 22 controls trigger device 12 such that control signals are transmitted in a substantially continuous fashion. According to another embodiment, processing circuit 22 controls trigger device 12 such that control signals are transmitted in an intermittent fashion. In yet further embodiments, processing circuit 22 controls trigger device 12 such that control signals are transmitted on an irregular basis, or alternatively, in response to user inputs (e.g., inputs received by way of input/output device 28).

In some embodiments, processing circuit 22 also controls the type of control signal transmitted. The control signal can be or include an electromagnetic wave (e.g., a radio wave, a microwave, infrared radiation, etc.), an acoustic wave, or other type of control signal (e.g., an ultrasonic signal, optical signal, magnetic field signal, etc.). Processing circuit 22 may also control operation of trigger device 12 to change various characteristics of a signal wave (e.g., frequency, amplitude, etc.). In alternative embodiments, the control signal may include data transmissions for receipt by release device 16 (e.g., to provide instructions to release device 16 regarding the amount, type, etc. of chemical substances to release).

Input/output device 28 may include a wide range of input/output devices, such as touchscreens, keyboards, microphones, speakers, etc. In one embodiment, device 28 is configured to enable a user to provide inputs to and receive inputs from detection system 11. In other embodiments device 28 is configured to receive data from and transmit data to various remote sources (e.g., remote computers, etc.). In one embodiment, device 28 is usable by, for example, security personnel to control operation of trigger device 12 and/or sensor device 14 via processing circuit 22.

Trigger device 12 generates control signal 20. As indicated earlier, control signal 20 may take various forms, and as such, the configuration of trigger device 12 may vary accordingly. In one embodiment, trigger device 12 is configured to provide electromagnetic waves, such as radio waves, microwaves, and the like. As such, trigger device 12 may include a wave generator and an antenna configured to generate and transmit various wave forms. According to an alternative embodiment, trigger device 12 is configured to provide acoustic waves. As such, trigger device 12 may include an acoustic wave generator such as a transducer or similar device. According to various other embodiments, trigger device may be configured to generate and/or transmit other types of control signals for receipt by release device 16 (e.g., ultrasonic, optical, magnetic, etc.).

Referring further to FIG. 2, release device 16 is shown in greater detail according to one embodiment, and includes substrate 30 and chamber 32. Substrate 30 is configured for attachment to attachment surface 36 (e.g., a surface of an object, a skin of a person, etc.). Chamber 32 serves as a storage and release device for substance 18 contained therein. Chamber 32 includes a closure member 34 configured to maintain chamber 32 in a sealed or closed configuration until an appropriate control signal is received, based upon which closure member 34 is configured to enable release of substance 18 to the surrounding environment. As discussed in greater detail below, closure member 34 may be provided in various forms, including various materials configured to degrade or rupture, a valve, and the like.

According to one embodiment, closure member 34 provides for a single release of substance 18. For example, closure member may be a rupturable portion of material (e.g., a sidewall of chamber 32). Alternatively, closure member 34 may be a thermally-sensitive material configured to degrade under elevated temperature conditions. As such, closure member 34 is in one embodiment configured to thermally degrade in response to a control signal to provide release of substance 18. In other embodiments, closure member 34 may include a valve or closable aperture configured to enable multiple releases of substance 18 from chamber 32

Referring to FIG. 3, release device 116 is shown according to one embodiment. As shown in FIG. 3, release device 116 includes controller 124 (e.g., a processor and memory, power source 117, one or more sensors 119, etc.) disposed on substrate 130 and coupled to chambers 132, 134, 136, which are configured to contain substances 118, 120, 122 respectively. Controller 124 is configured to control the release of substances 118, 120, 122 based on receipt of a control signal (e.g., a control signal provided by trigger device 12 or a similar device). As such, the control signal may include data regarding which substances are to be released, how much substance is to be released, the timing of the release of each substance, and the like. The release of substances 118, 120, 122 can be controlled using any of the closure members shown in FIGS. 4-6, or by another suitable device.

In some alternative embodiments, controller 124 is configured to release substances based on a cessation of a signal, based on a presence or absence of signals in a specific sequence/timeline, etc. For example, controller 124 is one embodiment configured to receive signals either continuously or intermittently (e.g., as an RF signal provided from a security beacon within a store). Should the release device (along with controller 124) be placed in an RF-shielded container (e.g., in an effort to avoid store security systems), controller 124 is configured to control the release of one or more of substances 118, 120, 122. As such, in addition, to controlling the release of substances 118, 120, 122 based on receipt of a control signal, similar control may be based on a cessation of the control signal, a change in a control signal pattern, timing, etc., and the like.

In some embodiments, controller 124 is or includes a power source or power supply (e.g., power source 117). The power source may be any suitable power source, including an electrical power storage device, a mechanical power storage device, a power transfer device, a transducer, and the like. In some embodiments, the power source includes a battery. In other embodiments, the power source includes a solar cell. In yet further embodiments, the power source is configured to receive and transfer power from other devices (e.g., by received energy in the form of wireless signals, heat, and the like). As such, in some embodiments, power source 117 is or includes an energy harvesting device configured to harvest energy from ambient sounds, motion, RF signals, and the like. Other types of power sources may be used according to various alternative embodiments.

As shown in FIG. 3, three chambers 132, 134, 136 are provided as part of release device 116, and each contains a different chemical substance. As such, based on a received control signal, up to 6 different substances, or combinations of substances, may be released by release device 116. In other embodiments, additional chambers may be utilized, thereby increasing the potential number of different substances and combinations of substances that can be released by release device 116. According to various alternative embodiments, one or more of chambers 132, 134, 136 include the same chemical substance.

In one embodiment, the release device (e.g., release device 16 or 116) may be or include a smart tag configured to determine whether to release one or more signals based on a variety of inputs, changes in condition , etc. (or the absence thereof). For example, release device 116 may be configured such that depending on an input or a sensed condition, any number of combinations of chemical substances may be released, each providing a unique chemical signature, or chemical signal. One or more sensors may sense information regarding or receive inputs for release device 116 and provide data to controller 124.

In some embodiments, controller 124 may be configured to selectively control the release of chemical substances from chambers 132, 134, 136 based on a variety of factors or conditions, including data received from sensors 119 or other sources. The factors may include receipt of a control signal, passage of a period of time, a current state or a change in motion, acceleration, altitude, pressure, temperature, an indication of tampering with the release device or an object to which the release device is operatively connected, and the like. Controller 124 may be configured to selectively release one or more chemical substances from chambers 132, 134, 136 based on a variety of other factors and conditions according to various other embodiments. These smart tag features may be implemented with any of the release devices disclosed herein.

Referring to FIG. 4, release device 216 is shown according to one embodiment. As shown in FIG. 4, release device 216 includes a substrate 230 and a wall portion 232 that form a chamber 234. Chamber 234 is configured to contain and provide controlled release of substance 218. According to one embodiment, chamber 234 is a thermally responsive member configured to enable release of substance 218 based on a control signal. For example, substrate 230 may be a resistive member or strip configured to increase in temperature in response to a control signal, thereby causing a corresponding increase in temperature of wall portion 232 and/or substance 218. In one embodiment, wall portion 232 is configured to thermally degrade at elevated temperatures, thereby providing release of substance 218. For example, wall portion 232 may include a microballoon printed over a resistive strip. In other embodiments, wall portion 232 may be or include a weakened area of material configured to rupture based upon expansion of substance 218 (e.g., a gas or volatile liquid) due to the elevated temperatures.

As shown in FIG. 4, a single chamber 234 is formed by substrate 230 and wall portion 232. In other embodiments, release device 216 may include multiple chambers having the same or different substances therein. Furthermore, the characteristics of the substrate or wall portion may be varied to enable different chambers to release substances at different times based on one or more control signals. For example, the resistivity of substrate 230 can be varied to provide for different changes in temperature based on receipt of control signals. Alternatively, different wall portions can be provided with different strength properties such that higher temperatures and/or greater internal temperatures may be required for release of a substance. Other ways of controlling the release of substances from chambers formed by substrate 230 and wall portion 232 can be used according to various alternative embodiments.

Referring to FIG. 5, release device 316 is shown according to one embodiment. As shown in FIG. 5, release device 316 includes a substrate including wells 332, 334 (e.g., recesses, pockets, etc.) formed therein. Wall portions 336, 338 are provided over wells 332, 334 respectively, to form chambers 333, 335. Chambers 333, 335 are configured to contain and provide controlled release of substances 318, 320. In one embodiment, substrate 330 is part of a microelectromechanical systems (MEMS) integrated circuit device or chip. In other embodiments, substrate 330 may take other forms.

Wall portions 336, 338 are in one embodiment configured to rupture or degrade to provide release of substances 318, 320. For example, wall portions 336, 338 may be configured to thermally degrade in response to temperature increases caused by one or more control signals.

In other embodiments, wall portions 336, 338 are or include weakened portions of material configured to rupture in response to increased internal pressures within chambers 333, 335 (e.g., due to expansion of gases resulting from increased temperatures, etc.).

As shown in FIG. 5, two chambers 333, 335 are formed in release device 316. In one embodiment, chambers 333, 335 include the same substances therein. In other embodiments, chambers 333, 335 include different substances. Furthermore, more or fewer chambers (e.g., 1, 3, etc.) may be used according to various alternative embodiments and the characteristics of the individual chambers (e.g., wall strength, etc.) can be varied to vary the release characteristics of the chambers.

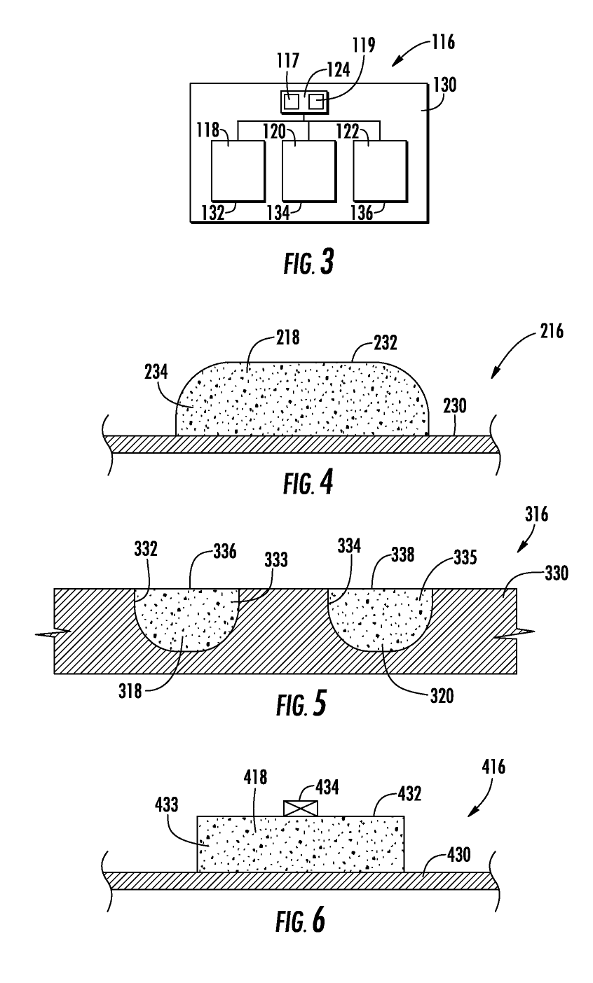

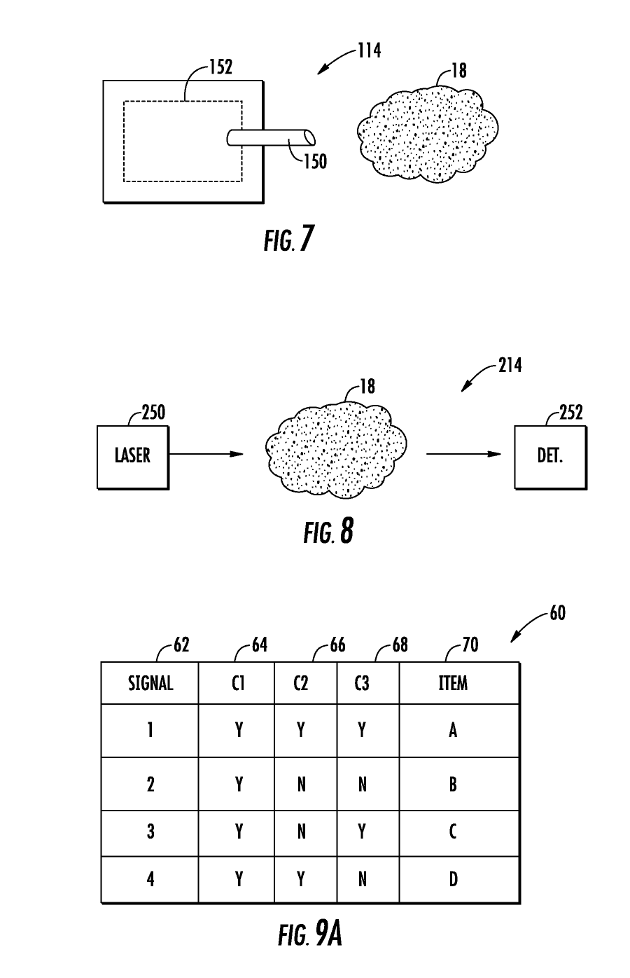

Referring to FIG. 6, release device 416 is shown according to one embodiment. Release device 416 includes substrate 430 and wall portion 432 that form chamber 433. Chamber 433 is configured to contain and provide controlled release of substance 418 based on receipt of one or more control signals. In one embodiment, chamber 433 includes a valve 434 configured to control release of substance 418 from chamber 433. Valve 434 is moveable, or actuatable, between a closed position and one or more open positions based on receipt of a control signal. According to one embodiment, valve 434 is an active valve actuatable based on data provided by a control signal. In another embodiment, valve 434 is a passive valve that is actuated based on excitation or stimulation provided by the control signal. For example, valve 434 may be a microvalve that includes a magnetostrictive resonator such that the resonator opens the valve when sufficiently stimulated by the control signal. Other types of valves can be used according to various other embodiments.

As detailed elsewhere herein, in some embodiments, the one or more of the release devices disclosed herein (e.g., release device 16, 116, 216, 316, 416, etc.) may include a controller or control system configured to control release of one or more chemical substances. The controller may be powered by a power source that may take a variety of forms, including a battery, solar cell, and the like. In further embodiments, the power source is configured to obtain energy from a control signal, a remote source (e.g., light, motion of a related object, etc.). In yet further embodiments, the power source may be or include a spring having stored energy that is usable to actuate a valve or other device based on various trigger events.

It should be understood that the release devices shown in FIGS. 3-6 are examples of release devices that may be utilized, and that other release devices may be used according to other exemplary embodiments. The chemical substance may be released as a vapor, aerosol, liquid or other form, and delivered by a vortex ring, a jet, in a pulsed fashion (e.g., based on a control signal), etc. Furthermore, the features of the different release devices illustrated herein may be used alone or in combination, such that a single release device may include any or all of the features shown in FIGS. 3-6 or elsewhere herein.

Referring now to FIG. 7, sensor device 114 is shown according to one embodiment. Sensor device 114 includes an intake member 150 (e.g., a flow tube, etc.) and an analyzer 152. Intake member 150 is configured to intake a fluid sample (e.g., gas or liquid) from a surrounding environment and provide the fluid sample to analyzer 152. Analyzer 152 is configured to identify one or more chemical substances 18 contained in the fluid sample. In one embodiment, device 114 is operable proximate a release device such as release device 16. In other embodiments, device 114 is operable remotely from the release device. Furthermore, intake member 150 may take any shape or form, and in some embodiments may provide a fluid transport mechanism for transporting a fluid sample from a remote location to analyzer 152.

In some embodiments, sensor device 114 is positioned within a fluid handling device or system (e.g., a ventilation air duct such as a return air duct, a fluid or liquid drain system, etc.). As such, in some embodiments intake member 150 may be or be part of one or more conduits or other devices used to direct fluid.

Analyzer 152 may be any device suitable for identifying one or more chemical substances within a fluid sample of a surrounding environment. For example, in one embodiment, analyzer 152 includes a mass spectrometer, a gas chromatograph, or a MEMS sensor array. In one embodiment, analyzer 152 includes a number of individual sensors that react in some way (e.g., experience a change in electrical properties, etc.) when in contact with certain chemical substances. Data from numerous sensors can be aggregated to form a chemical signature for a sample. The chemical signature in some embodiments is used as a chemical signal that may trigger other actions or be associated with various things, persons, etc. Other types of analysis devices can be used according to various other embodiments (e.g., biosensors, infrared sensors, etc.). Furthermore, analyzer 152 can include an integrated output device (e.g., a display, etc.), or alternatively, data regarding one or more substances (e.g., substance data) identified by analyzer 152 can be provided to input/output device 28 or a similar device for display or otherwise being provided to a user or another device.

In one embodiment, control circuit 22 controls the operation of sensor device 114. For example, control circuit 22 may control the timing of when fluid samples of a surrounding environment are taken based on one or more factors. In one embodiment, control circuit 22 directs sensor device 114 to sample the surrounding environment based on a user input received from, for example, input/output device 28. In other embodiments, control circuit 22 directs sensor device 114 to sample the surrounding environment a period of time after transmission of a control signal. In yet further embodiments, control circuit 22 directs sensor device 114 to sample the surrounding environment continuously, or at regular time intervals. According to various other alternative embodiments, the timing of sampling a surrounding environment can be controlled according to other factors or inputs.

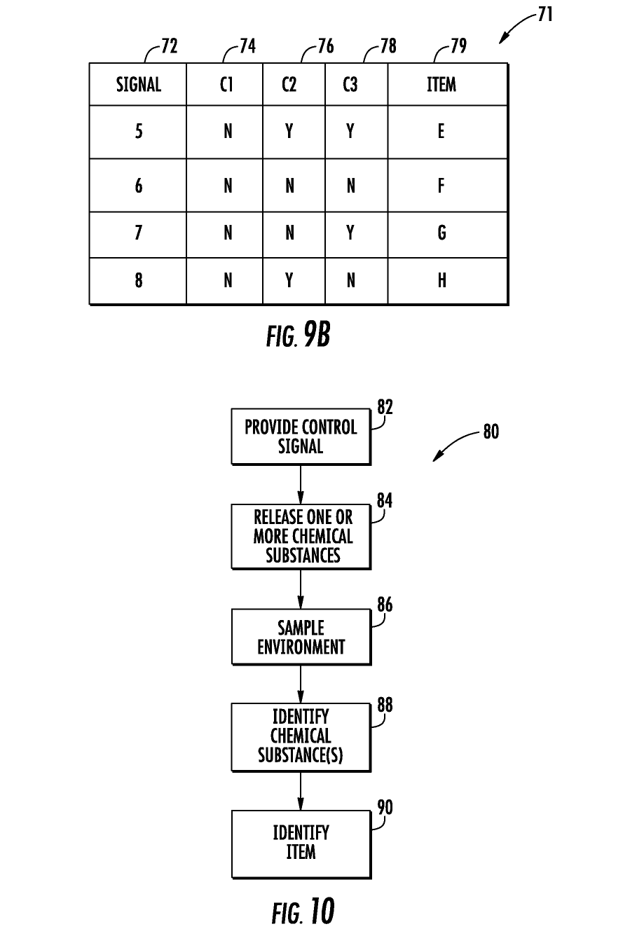

Referring to FIG. 8, sensor device 214 is shown according to one embodiment. As shown in FIG. 8, sensor device 214 includes laser 250 and detector 252. Sensor device 214 is configured to identify one or more chemical substances 18 in a surrounding environment. Laser 250 directs one or more laser pulses toward a fluid sample of a surrounding environment. The laser pulses interact with chemical substance 18 in the surrounding environment, scattering photons. Data regarding optical wavelengths absorbed, emitted, or scattered is captured by detector 252. Based on the captured data, one or more chemical substances can be identified. As with sensor device 114, various outputs can be provided to users, and the control of sensor device 214 can be based on a variety of factors. Sensor device 214 can include one or more technologies including differential infrared absorption lidar (DIAL), fluorescence spectroscopy, photoacoustic spectroscopy, and the like. Further, the laser may be scanned to provide location information (e.g., 1D, 2D, or 3D) for sensed chemicals.

Referring to FIG. 11, according to various alternative embodiments, various types of sensing devices may be utilized at various locations. For example, in some embodiments, a sensing device such as sensing device 14 may be positioned close to or proximate a release device such as release device 16. FIG. 11 shows a number of alternative positions for sensing device 14, or multiple sensing devices. Multiple sensing devices 14 may be utilized in order to establish a time or concentration/strength gradient for the release of substances from release device 16. For example, by capturing substance data regarding the time and presence of substances at a plurality of sensing devices 14 arranged along arrow 15, concentration and/or timing data associated with the release and presence of various substances can be established. Further, location information, such as a direction and distance to a release device, can be established based on substance data from various sensing devices. Any suitable number of sensing devices can be used, and the sensing devices can be arranged in any suitable configuration. Furthermore, as discussed elsewhere herein, the sensing devices can act upon a local environment (e.g., by obtaining a physical sample of a local fluid) or a remote environment (e.g., by using lasers, etc.). Thus, various sensing devices may be located proximate to/remotely from a release device and act on local and/or remote environments.

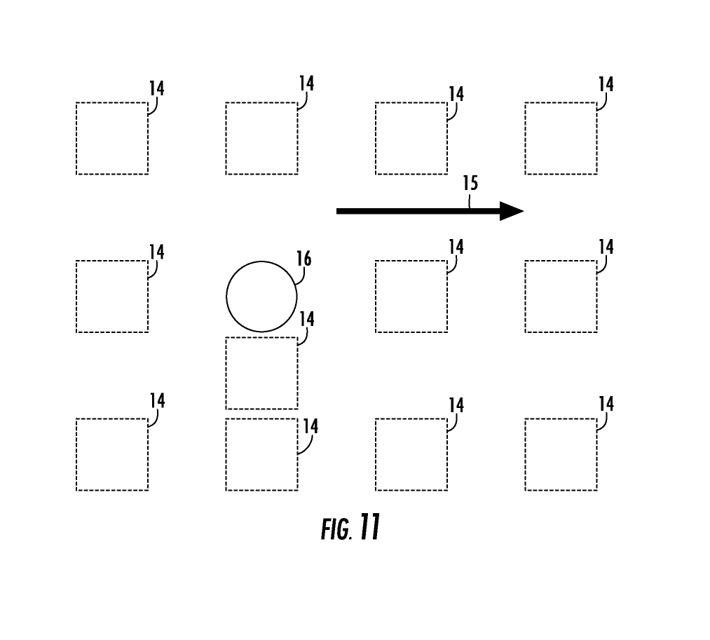

Referring now to FIG. 9A-9B, various data regarding control signals and chemical substances that may be released based on the transmission of various control signals to a release device is shown according to one embodiment. The data shown in FIG. 9A-9B may be stored by processing circuit 22 (e.g., in a computer database, etc.), on-board a release device such as release device 16, or in another storage device. As shown in FIG. 9A, different control signals 62 may be associated with chemical substances 64, 66, 68, and four different items 70. In one embodiment, a single release device can be configured for attachment to different items 70 (e.g., items A, B, C, D). The release device may be configured to release different chemical substances, or different combinations of chemical substances, based on the control signal (e.g., signals 1, 2, 3, and 4) or based on information sensed by or input to the release device. As such, based on the control signal transmitted, a specific chemical signature can be identified and associated with items 70. For example, upon transmission of the control signal designated signal 1, chemicals C1, C2, and C3 are released, corresponding to item A. The combinations shown in FIG. 9 are provided for illustration purposes only, and should not be construed as limiting.

As shown in FIG. 9A, chemical C1 is released in each instance. As shown in FIG. 9B, different control signals 72 may be associated with chemical substances 74, 76, 78, and four different items 79, without the release of chemical C1. In one embodiment, a single release device can be configured for attachment to different items 79 (e.g., items E, F, G, H). The release device may be configured to release different chemical substances, or different combinations of chemical substances, based on the control signal (e.g., signals 5, 6, 7, and 8) or based on information sensed by or input to the release device. As such, based on the control signal transmitted, a specific chemical signature can be identified and associated with items 79. For example, upon transmission of the control signal designated signal 5, chemicals C2 and C3 are released, corresponding to item E. The combinations shown in FIG. 9B are provided for illustration purposes only, and should not be construed as limiting.

It should be noted that various types of control signals may be used in combination with the trigger and release devices disclosed herein. Furthermore, the control signal may be encoded with data usable by the control device(s) to define the timing of release of chemicals, which chemicals to release, the amounts of chemicals to release, which device(s) of a group of devices should be triggered, and the like. For example, a trigger device may utilize any or all of signal frequency, duration, and/or modulation to encode instructions regarding the release of one or more chemicals. In some embodiments, the control signals and release device may be configured such that the release device is triggered directly based on the control signal (e.g., such that specific frequencies trigger resonant devices such as resonant MEMS, electrostrictive actuators, etc.). In other embodiments, the control signal is processed by a processing circuit such that the control signal is decoded and the appropriate chemicals released. The control signal may be provided in analog or digital form, including utilizing communication formats such as RFID, WiFi, or other RF data communications.

Referring to FIG. 10, method 80 of releasing and detecting chemical substances is shown according to one embodiment. A control signal is provided (82). The control signal may be provided by any suitable trigger device, such as trigger device 12 shown in FIG. 1. Based on the control signal, one or more chemical substances are released (84). The chemical substances may be released by a release device such as any of release devices 16, 116, 216, 316, or 416, or by other release devices. The chemical substances are released into a surrounding environment. A fluid sample of the surrounding environment is acquired (86) and one or more chemical substances are identified (88). The surrounding environment may be sampled using any of a variety of devices, including those discussed with respect to FIGS. 7-8. Based on identification of one or more chemical substances, an item can be identified (90). For example, as discussed with respect to FIG. 9, various chemical signatures can be associated with different items or persons, such that based on identification of one or more chemical substances, a corresponding item or person can be identified.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system for selective release of a chemical substance as a signal, comprising:
a release device configured to release a chemical substance into a surrounding environment based on receipt of a control signal;
a trigger device configured to transmit the control signal to the release device; and
a sensor device configured to detect the chemical substance in the surrounding environment.

2-4. (canceled)

5. The system of claim 1, wherein the release device includes a chamber defining an interior configured to store the chemical substance and a closure member configured to seal the interior from the surrounding environment.

6. The system of claim 5, wherein the closure member is configured to open to release the chemical substance from the interior based on receipt of the control signal.

7. The system of claim 6, wherein the closure member is configured to enable multiple releases of the chemical substance at different times.

8. The system of claim 6, wherein the closure member includes a thermally-responsive member configured to open based on receipt of the control signal.

9. The system of claim 6, wherein the closure member includes a valve.

10. The system of claim 9, wherein the valve is a magnetostrictive valve.

11. The system of claim 5, wherein the closure member includes at least one of a thermally degradable encapsulant and a chemically degradable encapsulant.

12-14. (canceled)

15. The system of claim 5, wherein the chamber includes a plurality of sub-chambers.

16. The system of claim 15, wherein each of the plurality of sub-chambers is configured to store a different one of a plurality of different chemical substances.

17. The system of claim 16, wherein less than all of the plurality of sub-chambers release different chemical substances to the surrounding environment based on receipt of the control signal.

18-21. (canceled)

22. The system of claim 1, wherein the control signal includes at least one of an ultrasonic signal, an optical signal, an acoustic wave, and a magnetic field signal.

23. (canceled)

24. The system of claim 1, wherein the control signal is encoded to provide an indication of at least one of a timing of release, an identification of the chemical to release, and an amount of the chemical to release.

25. The system of claim 1, wherein the control signal is encoded by way of at least one of signal frequency, signal duration, and signal modulation.

26-83. (canceled)

84. A system for identifying items, comprising:
a release device configured for attachment to an item and to release a chemical substance into a surrounding environment based on receipt of a control signal;
a trigger device configured to transmit the control signal to the release device;
a sensor device configured to sense the presence of the chemical substance in the surrounding environment and acquire substance data regarding the presence of the chemical substance in the surrounding environment; and
a processing circuit configured to identify the item based on the substance data.

85-86. (canceled)

87. The system of claim 84, wherein the release device includes a chamber defining an interior configured to store the chemical substance, and a closure member configured to seal the interior from the surrounding environment.

88. The system of claim 87, wherein the closure member is configured to open to release the chemical substance from the interior based on receipt of the control signal.

89. The system of claim 88, wherein the closure member is configured to enable multiple releases of the chemical substance at different times.

90. The system of claim 88, wherein the closure member includes a thermally-responsive member configured to open in response to the control signal.

91. The system of claim 88, wherein the closure member includes a valve.

92-96. (canceled)

97. The system of claim 87, wherein the chamber includes a plurality of sub-chambers.

98. The system of claim 97, wherein each sub-chamber is configured to store a different one of a plurality of different chemical substances.

99. The system of claim 98, wherein less than all of the plurality of sub-chambers release the different chemical substances to the surrounding environment based on receipt of the control signal.

100-103. (canceled)

104. The system of claim 84, wherein the control signal includes at least one of an ultrasonic signal, an optical signal, an acoustic wave, and a magnetic field signal.

105. (canceled)

106. The system of claim 104, wherein the control signal is encoded to provide an indication of at least one of a timing of release, an identification of the chemical to release, and an amount of the chemical to release.

107. The system of claim 106, wherein the control signal is encoded by way of at least one of signal frequency, signal duration, and signal modulation.

108-307. (canceled)

308. The system of claim 84, wherein the chemical substance is a gaseous substance.

309. The system of claim 308, wherein the sensor device is configured to sample the surrounding environment to detect the presence of the gaseous substance.

310. The system of claim 84, wherein the closure member includes at least one of a thermally degradable substance and a chemically degradable substance.

311. The system of claim 84, wherein the control signal is a wireless control signal including electromagnetic waves.

312. The system of claim 84, wherein the sensor device is configured to be positioned proximate the release device and detect the presence of the chemical substance in a remote environment.

313. The system of claim 84, wherein the sensor is configured to be positioned remote from the release device and detect the chemical substance in a local environment.

314. The system of claim 84, wherein the sensor is configured to be positioned remote from the release device and detect the chemical substance in a remote environment.
